(19) United States
(12) Patent Application Publication
(10) Pub. No.: US 2006/0281687 A1
(43) Pub. Date: Dec. 14, 2006

(54) Cyclin groove inhibitors

(76) Inventors: Martin J.I. Andrews, Dundee (GB); Campbell McInnes, Dundee (GB); George Kontopidis, Dundee (GB); Peter Martin Fischer, Angus (GB)

Correspondence Address: LAHIVE & COCKFIELD, 28 STATE STREET, BOSTON, MA, 02109, US

(21) Appl. No.: 11/408,905

(22) Filed: Apr. 21, 2006

Related U.S. Application Data
(63) Continuation of application No. PCT/GB04/04454, filed on Oct. 21, 2004.

(30) Foreign Application Priority Data
Oct. 21, 2003 (GB) 0324598.2

(51) Int. Cl. A61K 38/08; C07K 7/06

(52) U.S. Cl. 514/17; 530/330

(57) ABSTRACT

The present invention relates to a compound of formula I, or a variant thereof,
A-(B)ₘ-C-(D)ₙ-E (I) wherein m and n are each independently 0 or 1 and A, B, C, D and E are each independently linked to the respective adjacent residue by a linker group independently selected from carboxamide, reduced carboxamide, sulfonamide, imine, semicarbazone, oxime and ethanolamine;
A is (i) a natural or unnatural amino acid residue having a side chain comprising at least one H-bond acceptor moiety and at least one H-bond donor moiety, or a derivative thereof; or (ii) R(CO), wherein R is a C₁-C₂₄ hydrocarbyl group comprising at least one H-bond acceptor moiety and optionally one or more H-bond donor moieties, and where R optionally contains one or more heteroatoms selected from S, O, and N, and is optionally substituted by one or more substituents selected from halogen, OMe, CN, CF₃, and NO₂; each of B and D is independently an amino acid residue selected from arginine, 4-(guanidinyl)phenylalanine (4-(Gu)Phe), piperidinylglycine (PipGly), piperidinylalanine (PipAla), pyridinylalanine, histamine, N,N-(dimethyl) lysine (DMLys), citrulline, glutamine, serine, lysine, asparagine, isoleucine and alanine, or a derivative thereof; C is NH—X—CO, where X is a C₁-C₄ alkylene group substituted by a straight-chain or branched C₁-C₆ alkylene group, said C₁-C₆ alkylene group optionally containing a H-bond donor or H-bond acceptor moiety; E is (i) a natural or unnatural amino acid residue having an aryl or heteroaryl side chain, or a derivative thereof; or (ii) NHR′, where R′ is a C₁-C₂₄ hydrocarbyl group, optionally containing one or more heteroatoms selected from N, O, and S, and optionally comprising one or more H-bond acceptor or donor moieties; said hydrocarbyl group further comprising a pendent C₄-C₁₂ aryl or heteroaryl group, which itself may be optionally substituted by one or more substituents selected from a H-bond donor moiety, a H-bond acceptor moiety, a halogen, Me, Et, ⁱPr, CF₃, CN and NO₂; wherein at least one of A and E is other than a natural or unnatural amino acid residue when A, B, C, D and E are each linked to the respective adjacent residue by a carboxamide group.

### RELATED APPLICATIONS

This application is a continuation of PCT/GB2004/004454, filed on Oct. 21, 2004, which claims priority to GB 0324598.2, filed on Oct. 21, 2003. The entire contents of each of these applications are hereby incorporated herein by reference in their entirety.

### BACKGROUND TO THE INVENTION

We have previously disclosed (Zheleva, D. I. et al., PCT Int. Pat. Appl. Publ. WO 2001040142, Cyclacel Limited, UK) peptides capable of inhibiting the function of cyclin-dependent protein kinases (CDKs), particularly CDK2, by virtue of blocking a recognition site present in many cyclin subunits, particularly cyclins E and A, rather than the kinase subunit of the functionally competent CDK-cyclin enzyme complexes. This recognition site, which is used by CDK-cyclin complexes to recruit various regulatory and substrate proteins, is referred to as the cyclin groove (McInnes, C. et al., 2003, Curr. Med. Chem. Anti-Cancer Agents, 3, 57).

The present invention relates to novel peptidomimetic compounds comprising between three and five residues, which are capable of binding to the cyclin groove and inhibiting CDK function.

### STATEMENT OF INVENTION

A first aspect of the invention relates to a compound of formula I, or a variant thereof,
A-(B)ₘ-C-(D)n-E (I)
wherein m and n are each independently 0 or 1 and A, B, C, D and E are each independently linked to the respective adjacent residue by a linker group independently selected from carboxamide (CO—N or N—CO), reduced carboxamide (CH₂—N or N—CH₂), sulfonamide (SO₂—N or N—SO₂), imine (N═C or C═N), semicarbazone (NCONHN═C or C═NNHCON), oxime (O—N═C or C═N—O) and ethanolamine (C(OH)CH₂—N or N—CH₂C(OH));
A is
(i) a natural or unnatural amino acid residue having a side chain comprising at least one H-bond acceptor moiety and at least one H-bond donor moiety, or a derivative thereof; or
(ii) R(CO), wherein R is a C₁-C₂₄ hydrocarbyl group comprising at least one H-bond acceptor moiety and optionally one or more H-bond donor moieties, and where R optionally contains one or more heteroatoms selected from S, O, and N, and is optionally substituted by one or more substituents selected from halogen, OMe, CN, CF₃, and NO₂;
   each of B and D is independently an amino acid residue selected from arginine, 4-(guanidinyl)phenylalanine (4-(Gu)Phe), piperidinylglycine (PipGly), piperidinylalanine (PipAla), pyridinylalanine, histamine, N,N-(dimethyl) lysine (DMLys), citrulline, glutamine, serine, lysine, asparagine, isoleucine and alanine, or a derivative thereof;
   C is NH—X—CO, where X is a C₁-C₄ alkylene group substituted by a straight-chain or branched C₁-C₆ alkylene group, said C₁-C₆ alkylene group optionally containing a H-bond donor or H-bond acceptor moiety;
   E is
(i) a natural or unnatural amino acid residue having an aryl or heteroaryl side chain, or a derivative thereof; or
(ii) NHR′, where R′ is a C₁-C₂₄ hydrocarbyl group, optionally containing one or more heteroatoms selected from N, O, and S, and optionally comprising one or more H-bond acceptor or donor moieties; said hydrocarbyl group further comprising a pendent C₄-C₁₂ aryl or heteroaryl group, which itself may be optionally substituted by one or more substituents selected from a H-bond donor moiety, a H-bond acceptor moiety, a halogen, Me, Et, ⁱPr, CF₃, CN and NO₂;
   wherein at least one of A and E is other than a natural or unnatural amino acid residue when A, B, C, D and E are each linked to the respective adjacent residue by a carboxamide group.

A second aspect of the invention relates to a pharmaceutical composition comprising a compound of formula I admixed with a pharmaceutically acceptable diluent, excipient or carrier.

A third aspect of the invention relates to the use of a compound of formula I in the preparation of medicament for the treatment of a proliferative disorder.

A fourth aspect of the invention relates to an assay for identifying candidate substances capable of binding to a cyclin associated with a G1 control CDK enzyme and/or inhibiting said enzyme, comprising;
(a) bringing into contact a compound of formula I, said cyclin, said CDK and said candidate substance, under conditions wherein, in the absence of the candidate substance being an inhibitor of interaction of the cyclin/CDK interaction, the compound would bind to said cyclin, and
(b) monitoring any change in the expected binding of the compound and the cyclin.

A fifth aspect of the invention relates to an assay for the identification of compounds that interact with a cyclin or a cyclin when complexed with the physiologically relevant CDK, comprising:
(a) incubating a candidate compound and a compound of formula I, or a variant thereof, and a cyclin or cyclin/CDK complex,
(b) detecting binding of either the candidate compound or the compound with the cyclin.

### DETAILED DESCRIPTION

As used herein, the term “hydrocarbyl” refers to a group comprising at least C and H. If the hydrocarbyl group comprises more than one C then those carbons need not necessarily be linked to each other. For example, at least two of the carbons may be linked via a suitable element or group. Thus, the hydrocarbyl group may contain heteroatoms. Suitable heteroatoms will be apparent to those skilled in the art and include, for instance, sulphur, nitrogen, oxygen, phosphorus and silicon. Preferably, the hydrocarbyl group is an aryl, heteroaryl, alkyl, cycloalkyl, aralkyl or alkenyl group.

As used herein, the term “aryl” refers to a C6-12 aromatic group which may be substituted (mono- or poly-) or unsubstituted. Typical examples include phenyl and naphthyl etc.

As used herein, the term “heteroaryl” refers to a C₄₋₁₂ aromatic, substituted (mono- or poly-) or unsubstituted group, which comprises one or more heteroatoms. Preferred heteroaryl groups include pyrrole, pyrazole, pyrimidine, pyrazine, pyridine, quinoline, triazole, tetrazole, thiophene and furan.

As used herein, the term “alkyl” includes both saturated straight chain and branched alkyl groups which may be substituted (mono- or poly-) or unsubstituted. Preferably, the alkyl group is a C₁₋₂₀ alkyl group, more preferably a C₁₋₁₅, more preferably still a C₁₋₁₂ alkyl group, more preferably still, a C₁₋₆ alkyl group, more preferably a C₁₋₃ alkyl group. Particularly preferred alkyl groups include, for example, methyl, ethyl, propyl, isopropyl, butyl, isobutyl, tert-butyl, pentyl and hexyl.

The term “aralkyl” is used as a conjunction of the terms alkyl and aryl as given above.

As used herein, the term “cycloalkyl” refers to a cyclic alkyl group which may be substituted (mono- or poly-) or unsubstituted. Preferably, the cycloalkyl group is a C₃₋₁₂ cycloalkyl group.

As used herein, the term “alkenyl” refers to a group containing one or more carbon-carbon double bonds, which may be branched or unbranched, substituted (mono- or poly-) or unsubstituted. Preferably the alkenyl group is a C₂₋₂₀ alkenyl group, more preferably a C₂₋₁₅ alkenyl group, more preferably still a C₂₋₁₂ alkenyl group, or preferably a C₂₋₆ alkenyl group, more preferably a C₂₋₃ alkenyl group.

With regard to amino acid residues, as used herein the term “derivative thereof” refers to amino acids which are modified so that they are capable of forming a link to the adjacent residue by a linker group other than a carboxamide group, for example, by a reduced carboxamide, sulfonamide, imine, semicarbazone, oxime or ethanolamine group.

By way of illustration, amino acid residue B (shown below) with side chain R^{B} can be modified so as to link to the adjacent residue C (with side chain R^{C}) by a reduced carboxamide, sulfonamide, imine, semicarbazone, oxime or ethanolamine linker group.

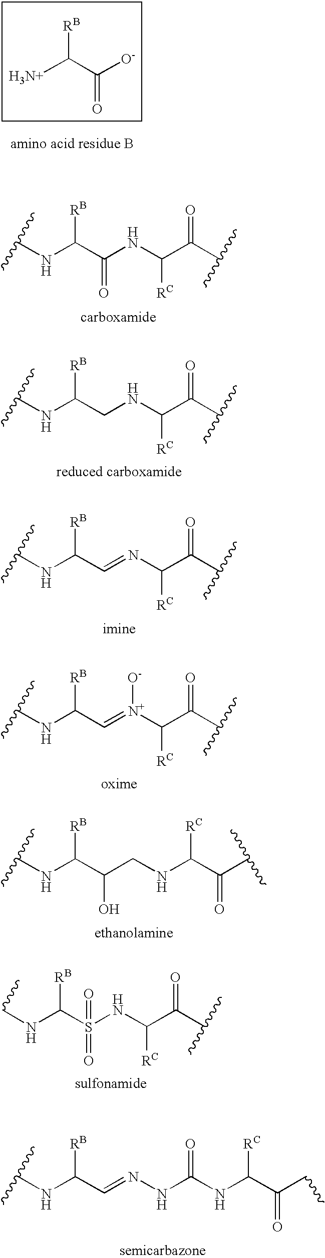

One preferred embodiment of the invention relates to a compound of formula Ia, or a variant thereof,
A-(B)ₘ-C-(D)ₙ-E (Ia)
wherein m and n are each independently 0 or 1 and A, B, C, D and E are each independently linked to the respective adjacent residue by a carboxamide (CO—N or N—CO), reduced carboxamide (CH₂—N or N—CH₂), sulfonamide (SO₂—N or N—SO₂), imine (N═C or C═N), semicarbazone (NCONHN═C or C═NNHCON), oxime (O—N═C or C═N—O) or ethanolamine (C(OH)CH₂—N or N—CH₂C(OH)) group;
A is
(i) a natural or unnatural amino acid residue having a side chain comprising at least one H-bond acceptor moiety and at least one H-bond donor moiety; or
(ii) R(CO), wherein R is a C₁-C₂₄ hydrocarbyl group comprising at least one H-bond acceptor moiety and optionally one or more H-bond donor moieties, and where R optionally contains one or more heteroatoms selected from S, O, and N, and is optionally substituted by one or more substituents selected from halogen, OMe, CN, CF₃, and NO₂;
   each of B and D is independently an amino acid residue selected from arginine, citrulline, glutamine, serine, lysine, asparagine, isoleucine and alanine;
   C is NH—X—CO, where X is a C₁-C₄ alkylene group substituted by a straight-chain or branched C₁-C₆ alkylene group, said C₁-C₆ alkylene group optionally containing a H-bond donor or H-bond acceptor moiety;
   E is
(i) a natural or unnatural amino acid residue having an aryl or heteroaryl side chain; or
(ii) NHR′, where R′ is a C₁-C₂₄ hydrocarbyl group, optionally containing one or more heteroatoms selected from N, O, and S, and optionally comprising one or more H-bond acceptor or donor moieties; said hydrocarbyl group further comprising a pendent C₄-C₁₂ aryl or heteroaryl group, which itself may be optionally substituted by one or more substituents selected from a H-bond donor moiety, a H-bond acceptor moiety, a halogen, Me, Et, ⁱPr, CF₃, CN and NO₂;
   wherein at least one of A and E is other than a natural or unnatural amino acid residue when A, B, C, D and E are each linked to the respective adjacent residue by a carboxamide group.

A more preferred embodiment of the invention relates to a compound of formula Ib, or a variant thereof,
A-(B)ₘ-C-(D)ₙ-E (Ib)
wherein m and n are each independently 0 or 1 and A, B, C, D and E are each independently linked to the respective adjacent residue by a carboxamide linker group (CO—NH or NH—CO);
A is
(i) a natural or unnatural amino acid residue having a side chain comprising at least one H-bond acceptor moiety and at least one H-bond donor moiety; or
(ii) R(CO), wherein R is a C₁-C₂₄hydrocarbyl group comprising at least one H-bond acceptor moiety and optionally one or more H-bond donor moieties, and where R optionally contains one or more heteroatoms selected from S, O, and N, and is optionally substituted by one or more substituents selected from halogen, OMe, CN, CF₃, and NO₂;
   each of B and D is independently an amino acid residue selected from arginine, 4-(guanidinyl)phenylalanine (4-(Gu)Phe), piperidinylglycine (PipGly), piperidinylalanine (PipAla), pyridinylalanine, histamine, N,N-(dimethyl) lysine (DMLys), citrulline, glutamine, serine, lysine, asparagine, isoleucine and alanine;
   C is NH—X—CO, where X is a C₁-C₄ alkylene group substituted by a straight-chain or branched C₁-C₆ alkylene group, said C₁-C₆ alkylene group optionally containing a H-bond donor or H-bond acceptor moiety;
   E is
(iii) a natural or unnatural amino acid residue having an aryl or heteroaryl side chain; or
(iv) NHR′, where R′ is a C₁-C₂₄ hydrocarbyl group, optionally containing one or more heteroatoms selected from N, O, and S, and optionally comprising one or more H-bond acceptor or donor moieties; said hydrocarbyl group further comprising a pendent C₄-C₁₂ aryl or heteroaryl group, which itself may be optionally substituted by one or more substituents selected from a H-bond donor moiety, a H-bond acceptor moiety, a halogen, Me, Et, ⁱPr, CF₃, CN and NO₂;
   providing that at least one of A and E is other than a natural or unnatural amino acid.

Thus, in one preferred embodiment of the invention, A, B, C, D and E are each linked to the respective adjacent residue by a carboxamide group, —CO—NH— or —NH—CO—.

Thus, in one preferred embodiment, the compound of the invention is of formula Ic, Id, Ie or If as shown below: 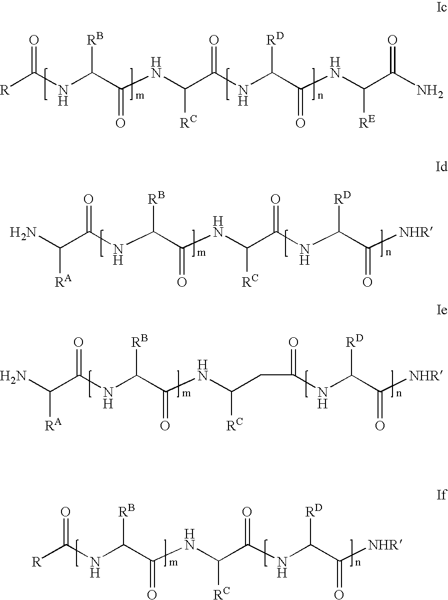
where R^{A}-R^{E} are the side chains of amino acid residues A-E respectively as defined above, and n, m R and R′ are as defined before.

In one preferred embodiment, the H-bond donor moiety is a functional group containing an N—H or O—H group, and the H-bond acceptor moiety is functional group containing C═O or N.

In a preferred embodiment, R optionally contains up to six heteroatoms, and is optionally substituted by up to six substituents selected from halogen, CN, CF₃, and NO₂.

Preferably, R is cycloalkyl, (CH₂O)ₓ-aryl or (CH₂O)ₓ-heteroaryl, and x is 0 or 1, wherein said cycloalkyl, aryl or heteroaryl group may be optionally substituted by one or more substituents selected from
NO₂;
halogen;
alkyl;
CF₃;
2-imidazolidinethione;
NH(CO)-heteroaryl, aryl or heteroaryl, each of which may be optionally substituted by one or more substituents selected from halogen, alkyl, NO₂, CF₃ and alkoxy.

In one particularly preferred embodiment, the heteroaryl group is selected from 1,2,4-triazole, benzothiazole, benzimidazole, pyrrole, isooxazole and imidazo[1,2-a]pyridine.

In one particularly preferred embodiment, A is a 1,2,4-triazole group optionally substituted with an alkyl or phenyl group, each of which may be optionally substituted by one or more halo, CF₃, NO₂ and/or alkoxy groups.

In another preferred embodiment, A is a benzothiazole group optionally substituted with one or more heteroaryl groups.

In more preferred embodiment, A is a benzothiazole group optionally substituted with one or more pyrrole groups.

In another preferred embodiment, A is an imidazo[1,2-a]pyridyl group optionally substituted with one or more alkyl and/or CF₃ groups.

In another preferred embodiment, A is a benzimidazole group optionally substituted with one or more halo and/or phenyl groups.

In another preferred embodiment, A is a cycloalkyl group optionally substituted with one or more CONH-heteroaryl substituents. More preferably, A is a cyclohexyl group optionally substituted with one or more CONH-heteroaryl substituents, wherein the heteroaryl substituent is an isooxazole group optionally substituted with one or more alkyl groups.

In another preferred embodiment, A is a phenyl group optionally substituted with one or more substituents selected from NO₂, 1,2,4-triazole and 2-imidazolidinethione.

In one preferred embodiment, A is selected from the following:

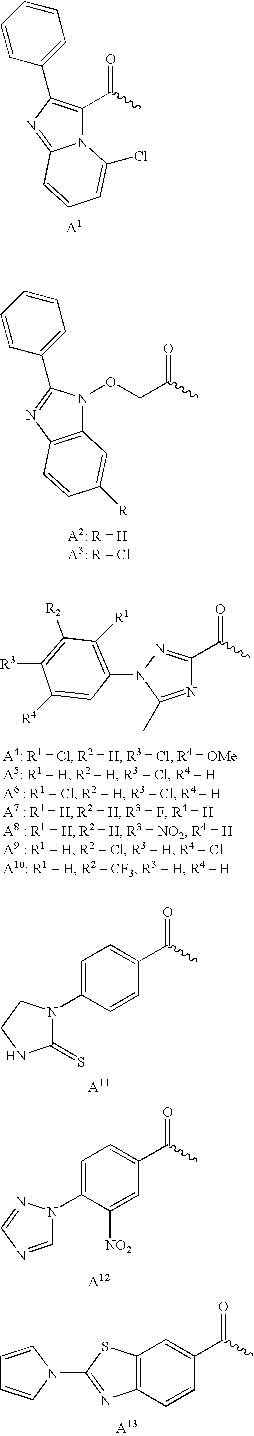

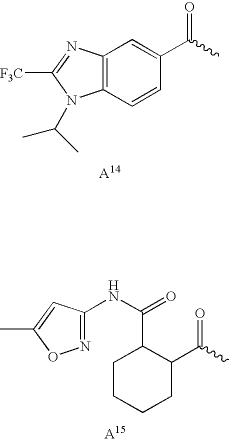

In one preferred embodiment, the hydrocarbyl group of E optionally contains up to six heteroatoms, and optionally comprises up to two H-bond acceptor or donor moieties, wherein the pendant C₁-C₁₂ aryl or heteroaryl group is optionally substituted by up to four substituents selected from a H-bond donor moiety, a H-bond acceptor moiety, a halogen, Me, Et, ⁱPr, CF₃, CN and NO₂.

In one particularly preferred embodiment, E is NHR′ and R′ is [CH(R^{a})CH₂NH]ₚ[CH₂]_{q}Ar^{a}[CH₂]ᵣAr^{b}, where R^{a} is a straight or branched chain C₁-C₆ alkyl group, p, q and r are each independently 0 or 1, and Ar^{a} and Ar^{b} are each independently aryl groups optionally substituted by one or more substituents selected from halogen, Me, Et, ⁱPr, CF₃, CN and NO₂.

In an even more preferred embodiment, E is 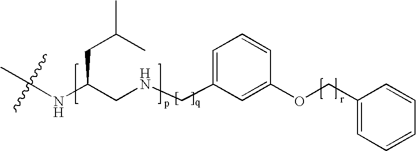
and p, q and r are each independently 0 or 1.

More preferably still, E is selected from the following:

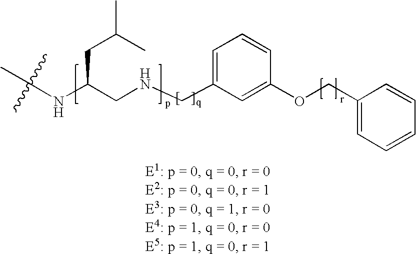

In a preferred embodiment, C is selected from alanine, valine, leucine, β-leucine, β-OH-β-leucine, isoleucine, aspartate, glutamate, asparagine, glutamine, lysine, arginine, serine and threonine.

Even more preferably, C is selected from leucine, isoleucine, β-leucine, β-OH-β-leucine, and asparagine;

More preferably still, C is leucine or β-leucine.

In a preferred embodiment, B is selected from arginine, 4-(guanidinyl)phenylalanine (4-(Gu)Phe), piperidinylglycine (PipGly), piperidinylalanine (PipAla), pyridinylalanine, histamine, N,N-(dimethyl) lysine (DMLys), citrulline, glutamine, serine and lysine.

In a more preferred embodiment, B is selected from arginine, 4-(guanidinyl)phenylalanine (4-(Gu)Phe), piperidinylglycine (PipGly), piperidinylalanine (PipAla), N,N-(dimethyl) lysine (DMLys), and lysine.

Even more preferably, B is arginine.

In a preferred embodiment, D is selected from asparagine, isoleucine and alanine.

Even more preferably, D is asparagine.

In another preferred embodiment, A is selected from arginine, glutamine, citrulline.

More preferably, A is arginine.

In another particularly preferred embodiment, E is selected from phenylalanine, para-fluorophenylalanine, meta-fluorophenylalanine, ortho-chlorophenylalanine, para-chlorophenylalanine, meta-chorophenylalanine, thienylalanine, N-methylphenylalanine, homophenylalanine (Hof), tyrosine, tryptophan, 1-naphthylalanine (1Nal), 2-naphthylalanine (2Nal) and biphenylalanine (Bip) or (Tic).

More preferably still, E is selected from phenylalanine, para-fluorophenylalanine, meta-fluorophenylalanine, ortho-chlorophenylalanine, para-chlorophenylalanine, meta-chorophenylalanine, thienylalanine and N-methylphenylalanine.

Even more preferably, E is para-fluorophenylalanine

Preferably, the variants involve the replacement of an amino acid residue by one or more, preferably one, of those selected from the residues of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

Such variants may arise from homologous substitution i.e. like-for-like substitution such as basic for basic, acidic for acidic, polar for polar etc. Non-homologous substitution may also occur i.e. from one class of residue to another or alternatively involving the inclusion of unnatural amino acids such as ornithine, diaminobutyric acid, norleucine, pyridylalanine, thienylalanine, naphthylalanine and phenylglycine.

As used herein, amino acids are classified according to the following classes;
basic; H, K, R
acidic; D, E
non-polar; A, F, G, I, L, M, P, V, W
polar; C, N, Q, S, T, Y,
   (using the internationally accepted amino acid single letter codes) and homologous and non-homologous substitution is defined using these classes. Thus, homologous substitution is used to refer to substitution from within the same class, whereas non-homologous substitution refers to substitution from a different class or by an unnatural amino acid.

The variants may also arise from replacement of an amino acid residue by an unnatural amino acid residue that may be homologous or non-homologous with that it is replacing. Such unnatural amino acid residues may be selected from;-alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid^{#}, 7-amino heptanoic acid*, L-methionine sulfone^{#}*, L-norleucine*, L-norvaline*, p-nitro-L-phenylalanine*, L-hydroxyproline^{#}, L-thioproline*, methyl derivatives of phenylalanine (Phe) such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation * has been utilised for the purpose of the discussion above, to indicate the hydrophobic nature of the derivative whereas# has been utilised to indicate the hydrophilic nature of the derivative, #* indicates amphipathic characteristics. The structures and accepted three letter codes of some of these and other unnatural amino acids are given in the Examples section.

One preferred embodiment relates to a variant of a compound according to the invention wherein:
(a) A is unchanged or conservatively substituted;
(b) B is substituted by any amino acid capable of providing at least one site for participating in hydrogen bonding;
(c) C is unchanged or conservatively substituted;
(d) D is unchanged or conservatively substituted;
(e) E is unchanged or substituted by any aromatic amino acid.

In one preferred embodiment of the invention, A is a natural or unnatural amino acid as defined above in which the NH2 group is acylated.

In another preferred embodiment, the invention relates to a compound of formula I, or variant thereof, which is (a) modified by substitution of one or more, preferably one, natural or unnatural amino acid residues by the corresponding D-stereomer; (b) a chemical derivative of the compound; (c) a cyclic compound derived from the compound of formula I or from a derivative thereof; (d) a dual compound; (e) a multimer of said compounds; (f) any of said compounds in the D-stereomer form; or (g) a compound in which E is natural or unnatural amino acid residue as defined above, and the order of D and E is reversed.

As used herein, the term “substitution” is used as to mean “replacement” i.e. substitution of an amino acid residue means its replacement.

The three letter notations appearing above are in accordance with IUPAC convention. The structure of various unnatural amino acid derivatives are provided in the introduction to the Examples, further expansion on nomenclature being given above.

The compounds of the present invention may be subjected to a further modification that is beneficial in the context of the present invention being conversion of the free carboxyl group of the carboxy terminal amino acid residue (when E is a natural or unnatural amino acid as defined above), to a carboxamide group. Thus, the C-terminal amino acid residue may be in the form —C(O)—NR^{x}R^{Y}, wherein R^{x} and R^{y} are each independently selected from hydrogen, C₁₋₆ alkyl, C₁₋₆ alkylene or C₁₋₆ alkynyl (collectively referred to “alk”), aryl such as benzyl or alkaryl, each optionally substituted by heteroatoms such as O, S or N. Preferably at least one of R^{x} or R^{y} is hydrogen, most preferably, they are both hydrogen. Thus, the present invention therefore encompasses compounds in which the C-terminal amino acid residue is in the carboxyl or carboxamide form.

In one preferred embodiment of the invention, m and n are both 1, i.e. compounds of formula A-B-C-D-E.

In another preferred embodiment of the invention, m is 1 and n is 0, i.e. compounds of formula A-B-C-E (i.e. where D is absent).

In another preferred embodiment, m is 0 and n is 1, i.e. compounds of formula A-C-D-E (i.e where B is absent).

In yet another preferred embodiment, m and n are both 0, i.e. compounds of formula A-C-E (where B and D are absent).

In one especially preferred embodiment, the compound is selected from the following:

| Compound | | | | | | |
|---|---|---|---|---|---|---|
| No. | N-terminus | | | | | C-terminus |
| 1 | A¹ | Arg | Leu | Asn | p-F-Phe | NH₂ |
| 2 | A⁴ | Arg | Leu | Asn | p-F-Phe | NH₂ |
| 3 | A⁵ | Arg | Leu | Asn | p-F-Phe | NH₂ |
| 4 | A⁶ | Arg | Leu | Asn | p-F-Phe | NH₂ |
| 5 | A¹¹ | Arg | Leu | Asn | p-F-Phe | NH₂ |
| 6 | A⁷ | Arg | Leu | Asn | p-F-Phe | NH₂ |
| 7 | A⁸ | Arg | Leu | Asn | p-F-Phe | NH₂ |
| 8 | A¹² | Arg | Leu | Asn | p-F-Phe | NH₂ |
| 9 | A² | Arg | Leu | Asn | p-F-Phe | NH₂ |
| 10 | A⁹ | Arg | Leu | Asn | p-F-Phe | NH₂ |
| 11 | A³ | Arg | Leu | Asn | p-F-Phe | NH₂ |
| 12 | A¹³ | Arg | Leu | Asn | p-F-Phe | NH₂ |
| 13 | A¹⁴ | Arg | Leu | Asn | p-F-Phe | NH₂ |
| 14 | A¹⁰ | Arg | Leu | Asn | p-F-Phe | NH₂ |

| | | | | | |
|---|---|---|---|---|---|
| 15 | A¹⁵ | Leu | Asn | p-F-Phe | NH₂ |

| | | | | | |
|---|---|---|---|---|---|
| 16 | A⁹ | Arg | βLeu | p-F-Phe | NH₂ |
| 17 | A⁹ | Lys | βLeu | p-F-Phe | NH₂ |
| 18 | A⁹ | 4-(Gu) Phe | βLeu | p-F-Phe | NH₂ |
| 19 | A⁹ | DMLys | βLeu | p-F-Phe | NH₂ |
| 20 | A⁹ | PipAla | βLeu | p-F-Phe | NH₂ |
| 21 | A⁹ | PipGly | βLeu | p-F-Phe | NH₂ |
| 22 | A⁹ | PipGly | βLeu | p-F-Phe | NH₂ |
| 23 | A⁹ | PipGly | βLeu | p-F-Phe | NH₂ |

| | | | | | |
|---|---|---|---|---|---|
| 24 | H | Arg | Arg | Leu | E¹ |
| 25 | H | Arg | Arg | Leu | E² |
| 26 | H | Arg | Arg | Leu | E³ |
| 27 | H | Arg | Arg | βLeu | E¹ |
| 28 | H | Arg | Arg | βLeu | E² |

| | | | | |
|---|---|---|---|---|
| 29 | H | Arg | Arg | E⁴ |
| 30 | H | Arg | Arg | E⁵ |

wherein A¹⁻¹⁵ and E¹⁻⁵ are as defined above and wherein each residue is linked to the adjacent residue via a carboxamide linker group.

Another preferred embodiment relates to a variant of a compound according to the invention, which is (a) modified by substitution of one or more natural or unnatural amino acid residues by the corresponding D-stereomer; (b) a chemical derivative of the compound; (c) a cyclic compound derived from the compound or derivative thereof; (d) a multimer of said compounds; (e) the D-stereomer form of said compound; or (f) a compound wherein the order of the final two residues at the C-terminal end are reversed.

Pharmaceutical Composition

A second aspect relates to a pharmaceutical composition comprising a compound according to the invention admixed with a pharmaceutically acceptable diluent excipient or carrier. Even though the compounds of the present invention (including their pharmaceutically acceptable salts, esters and pharmaceutically acceptable solvates) can be administered alone, they will generally be administered in admixture with a pharmaceutical carrier, excipient or diluent, particularly for human therapy. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Salts/Esters

The compounds of formula I can be present as salts or esters, in particular pharmaceutically acceptable salts or esters.

Pharmaceutically acceptable salts of the compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with amino acids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

Enantiomers/Tautomers

In all aspects of the present invention previously discussed, the invention includes, where appropriate all enantiomers and tautomers of compounds of formula I. The man skilled in the art will recognise compounds that possess an optical properties (one or more chiral carbon atoms) or tautomeric characteristics. The corresponding enantiomers and/or tautomers may be isolated/prepared by methods known in the art.

Stereo and Geometric Isomers

Some of the compounds of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those agents, and mixtures thereof. The terms used in the claims encompass these forms, provided said forms retain the appropriate functional activity (though not necessarily to the same degree).

The present invention also includes all suitable isotopic variations of the agent or pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ^{1.4}C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

Solvates

The present invention also includes the use of solvate forms of the compounds of the present invention. The terms used in the claims encompass these forms.

Polymorphs

The invention furthermore relates to the compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

Prodrugs

The invention further includes the compounds of the present invention in prodrug form. Such prodrugs are generally compounds of formula I wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

Administration

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 250 mg and more preferably from 10-100 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-250 mg, of active ingredient per dose.

Compositions may be formulated in unit dosage form, i.e., in the form of discrete portions containing a unit dose, or a multiple or sub-unit of a unit dose.

Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from 0.01 to 30 mg/kg body weight, such as from 0.1 to 10 mg/kg, more preferably from 0.1 to 1 mg/kg body weight.

In an exemplary embodiment, one or more doses of 10 to 150 mg/day will be administered to the patient.

Combinations

In a particularly preferred embodiment, the one or more compounds of the invention are administered in combination with one or more other therapeutically active agents, for example, existing drugs available on the market. In such cases, the compounds of the invention may be administered consecutively, simultaneously or sequentially with the one or more other active agents.

By way of example, it is known that anticancer drugs in general are more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining chemotherapeutic drugs are that it may promote additive or possible synergistic effects through biochemical interactions and also may decrease the emergence of resistance in early tumour cells which would have been otherwise responsive to initial chemotherapy with a single agent. An example of the use of biochemical interactions in selecting drug combinations is demonstrated by the administration of leucovorin to increase the binding of an active intracellular metabolite of 5-fluorouracil to its target, thymidylate synthase, thus increasing its cytotoxic effects.

Numerous combinations are used in current treatments of cancer and leukemia. A more extensive review of medical practices may be found in “Oncologic Therapies” edited by E. E. Vokes and H. M. Golomb, published by Springer.

Beneficial combinations may be suggested by studying the growth inhibitory activity of the test compounds with agents known or suspected of being valuable in the treatment of a particular cancer initially or cell lines derived from that cancer. This procedure can also be used to determine the order of administration of the agents, i.e. before, simultaneously, or after delivery. Such scheduling may be a feature of all the cycle acting agents identified herein.

Therapeutic Use

A third aspect relates to the use of a compound according to the invention in the preparation of medicament for the treatment of proliferative disorders such as cancers and leukaemias where inhibition of CDK2 would be beneficial.

As used herein the phrase “preparation of a medicament” includes the use of a compound of formula I directly as the medicament in addition to its use in a screening programme for further therapeutic agents or in any stage of the manufacture of such a medicament.

Such preparation, including their use in assays for identifying further candidate compounds, is described herein. The embodiments described as being preferred in the context of the compounds of the invention apply equally to their use.

In one preferred embodiment of the invention, the compound of formula I is capable of binding to the cyclin binding groove of a CDK enzyme. More preferably, the CDK enzyme is CDK2 or CDK4.

In one particularly preferred embodiment, the compound of formula I is capable of binding to the CDK2/cyclin A complex, as measured by a competitive binding assay. Further details of this assay may be found in the accompanying examples. Preferably, the compound of formula I exhibits an IC₅₀ value in the above-described competitive binding assay of less than 50 μM, more preferably less than 25 μM, more preferably less than 10 μM or 5 μM, more preferably still less than 1 μM, even more preferably less than 0.1 μM.

In another particularly preferred embodiment, the compound of formula I is capable of inhibiting CDK2/cyclin A as measured by a functional kinase assay. Further details of this assay may be found in the accompanying examples. Preferably, the compound of formula I exhibits an IC₅₀ value in the above-described functional kinase assay of less than 50 μM, more preferably less than 25 μM, more preferably less than 10 μM or 5 μM, more preferably still less than 1 μM, even more preferably less than 0.1 μM.

Assays

A further embodiment of the present invention relates to assays for candidate substances that are capable of modifying the cyclin interaction with CDKs, especially CDK2 and CDK4. Thus, such assays may involve incubating a candidate substance with a cyclin and a compound of the invention and detecting either the candidate-cyclin complex or free (unbound) compound of the invention. An example of the latter would involve the compound of the invention being labelled such as to emit a signal when bound to a CDK. The reduction in said signal being indicative of the candidate substance binding to, or inhibiting compound-cyclin interaction.

Suitable candidate substances include peptides, especially of from about 5 to 30 or 10 to 25 amino acids in size, based on the sequence of the various domains of p2 1, or variants of such peptides in which one or more residues have been substituted. Peptides from panels of peptides comprising random sequences or sequences which have been varied consistently to provide a maximally diverse panel of peptides may be used.

Suitable candidate substances also include antibody products (for example, monoclonal and polyclonal antibodies, single chain antibodies, chimeric antibodies and CDR-grafted antibodies) which are specific for p21 or cyclin binding regions thereof. Furthermore, combinatorial libraries, single-compound collections of synthetic or natural organic molecules, peptide and peptide mimetics, defined chemical entities, oligonucleotides, and natural product libraries may be screened for activity as modulators of cyclin/CDK/regulatory protein complex interactions in assays such as those described below. The candidate substances may be used in an initial screen in batches of, for example, 10 substances per reaction, and the substances of those batches which show inhibition tested individually. Candidate substances which show activity in in vitro screens such as those described below can then be tested in whole cell systems, such as mammalian cells.

Another aspect relates to an assay for identifying candidate substances capable of binding to a cyclin associated with a G1 control CDK enzyme and/or inhibiting said enzyme, comprising;
(a) bringing into contact a compound of formula I as defined above, said cyclin, said CDK and said candidate substance, under conditions wherein, in the absence of the candidate substance being an inhibitor of interaction of the cyclin/CDK interaction, the compound would bind to said cyclin, and
(b) monitoring any change in the expected binding of the compound and the cyclin.

Yet another aspect relates to an assay for the identification of compounds that interact a cyclin or a cyclin when complexed with the physiologically relevant CDK, comprising:
(a) incubating a candidate compound and a compound of formula I as defined above, or a variant thereof, and a cyclin or cyclin/CDK complex,
(b) detecting binding of either the candidate compound or the compound with the cyclin.

Preferably, the cyclin is selected from cyclin A, cyclin E or cyclin D.

Even more preferably, the cyclin is cyclin A.

In a preferred embodiment, the assay comprises the use of a three dimensional model of a cyclin and a candidate compound.

Preferably, at least one of the assay components is bound to a solid phase.

Preferably, the compound is labelled so as to emit a signal when bound to said cyclin.

Even more preferably, the cyclin is labelled so as to emit a signal when bound to the compound.

In one particularly preferred embodiment, one of the assay components is labelled with a fluorescence emitter and the signal is detected using fluorescence polarisation techniques.

A further aspect of the invention relates to a method of using a cyclin in a drug screening assay comprising:
(a) selecting a candidate compound by performing rational drug design with a three-dimensional model of said cyclin, wherein said selecting is performed in conjunction with computer modeling;
(b) contacting the candidate compound with the cyclin; and
(c) detecting the binding of the candidate compound for the cyclin groove; wherein a potential drug is selected on the basis of its having a greater affinity for the cyclin groove than that of a compound of formula I as defined above.

In a preferred embodiment, the method of detection comprises monitoring G0 and/or G1/S cell cycle, cell cycle-related apoptosis, suppression of E2F transcription factor, hypophosphorylation of cellular pRb, or in vitro anti-proliferative effects.

The assays of the present invention (discussed hereinafter with reference to cyclin A) encompass screening for candidate compounds that bind a cyclin “recruitment centre” or “cyclin groove” discussed above in respect of the prior art but herein defined in greater detail with reference to the amino acid sequence of preferably human cyclin A or of partially homologous and functionally equivalent mammalian cyclins. The substrate recruitment site from previously described cyclin A/peptide complexes consists mainly of residues of the α1 (particularly residues 207-225) and α3 (particularly residues 250-269) helices, which form a shallow groove on the surface, comprised predominantly of hydrophobic residues. This is discussed in greater detail in Russo AA et al. (Nature (1996) 382,325-331) with respect to p27/cyclin A. From the X-ray structure assigned to the p27/cyclin A/CDK2 provided therein it is possible to conclude that the sequence SACRNLFG of p27 that interacts with cyclin A does so through the following interactions cyclin A:

| p27 residue | Cyclin A residues |
|---|---|
| S | E220, E224 |
| A | W217, E220, V221, E224, I281 |
| C | Y280, I281, D283 |
| R | D216, W217, E220, Q254 |
| N | Q254, T285, Y286 |
| L | I213, L214, W217, Q254 |
| F | M210, I213, R250, G251, |
| | K252, L253, Q254 |
| G | T285 |

These residues are largely conserved in the A, B, E and D1 cyclins.

Previous studies by the applicant on p21 peptides (Zheleva, D. I. et al., PCT Int. Pat. Appl. Publ. WO 2001040142, Cyclacel Limited, UK) revealed that further distinct amino acid residues of cyclin A are important in the interaction between cyclin A and p21, especially with respect to the inhibitory activity of the peptides against CDK2. The cyclin A amino acids believed to be important for interaction with above-mentioned p21 peptides include:

| | Cyclin A residues | | |
|---|---|---|---|
| | Major | Intermediate | Minor |
| p21 residue | Interaction | Interaction | Interaction |
| H | E223, E224 | W217, V219, V221 | G222, Y225, I281 |
| | | S408, E411 | |
| A | | Y225 | E223 |
| K | D284 | | E220, V279 |
| R | | I213 | A212, V215, L218 |
| | | | Q406, S408 |
| R | D283 | I213, L214 | M210, L253 |
| L | L253 | G257 | L218, I239, V256 |
| I | | R250, Q254 | |
| F | I206, R211 | T207, L214 | M200 |

The present invention therefore includes assays for candidate compounds that interact with cyclin A by virtue of forming associations with at least two of the amino acid residues L253, 1206 and R211 of cyclin A or the corresponding homologous amino acids of cyclin D or cyclin E.

In a further preferred assay, the candidate compound may form associations with at least E223, E224, D284, D283, L253, I206 and R211 of cyclin A or the corresponding homologous amino acids of cyclin D or cyclin E.

In a preferred assay, the candidate compound may form further associations with W217, V219, V221, S408, E411, Y225, I213, L214, G257, R250, Q254, T207 and L214 of cyclin A or the corresponding homologous amino acids of cyclin D or cyclin E.

In a more preferred assay, the candidate compound may form further associations with G222, Y225, I281, E223, E220, V279, A212, V215, L218, Q406, S408, M210, L253, L218, I239, V256 and M200 of cyclin A or the corresponding homologous amino acids of cyclin D or cyclin E.

As used in this context the phrase “forming associations” is used to include any form of interaction a binding peptide may make with a ligand. These include electrostatic interactions, hydrogen bonds, or hydrophobic/lipophilic interactions through Van der Waals's forces or aromatic stacking, etc.

Also, as used herein in the context of assays of the present invention, the term “cyclin” is used to refer to cyclin A, cyclin D or cyclin E, or regions thereof that incorporate the “cyclin groove” as hereinbefore described. Thus, an assay may be performed in accordance with the present invention if it utilises the a full length cyclin protein or a region sufficient to allow the cyclin groove to exist, for example amino acids 173-432 or 199-306 of human cyclin A.

Thus, by utilising the compounds of the present invention especially those of the preferred embodiments in competitive binding assays with candidate compounds, further compounds that interact at this site may be identified and assigned utility in the control of the cell cycle by virtue of controlling, preferably inhibiting CDK2 and/or CDK4 activity. Such assays may be performed in vitro or virtually i.e. by using a three dimensional model or preferably, a computer generated model of a complex of a peptide of the present invention and cyclin A. Using such a model, candidate compounds may be designed based upon the specific interactions between the compounds of the present invention and cyclin A, the relevant bond angles and orientation between those components of the compounds of the present invention that interact both directly and indirectly with the cyclin groove.

As used herein the term “three dimensional model” includes both crystal structures as determined by X-ray diffraction analysis, solution structures determined by nuclear magnetic resonance spectroscopy as well as computer generated models. Such computer generated models may be created on the basis of a physically determined structure of a compound of the present invention bound to cyclin A or on the basis of the known crystal structure of cyclin A, modified (by the constraints provided by the software) to accommodate a compound of formula I. Suitable software suitable of the generation of such computer generated three dimensional models include AFFINITY, CATALYST and LUDI (Molecular Simulations, Inc.).

Such three dimensional models may be used in a program of rational drug design to generate further candidate compounds that will bind to cyclin A. As used herein the term “rational drug design” is used to signify the process wherein structural information about a ligand-receptor interaction is used to design and propose modified ligand candidate compounds possessing improved fit with the receptor site in terms of geometry and chemical complementarity and hence improved biological and pharmaceutical properties, such properties including, e.g., increased receptor affinity (potency) and simplified chemical structure. Such candidate compounds may be further compounds or synthetic organic molecules.

Using techniques known in the art, crystal or solution structures of cyclin A bound to a compound of the present invention may be generated, these too may be used in a programme of rational drug design as discussed above.

Crystals of the compounds of the present invention complexed with cyclin A can be grown by a number of techniques including batch crystallization, vapour diffusion (either by sitting drop or hanging drop) and by microdialysis. Seeding of the crystals in some instances is required to obtain X-ray quality crystals. Standard micro and/or macro seeding of crystals may therefore be used.

Once a crystal of the present invention is grown, X-ray diffraction data can be collected. Crystals can be characterized by using X-rays produced in a conventional source (such as a sealed tube or a rotating anode) or using a synchrotron source. Methods of characterization include, but are not limited to, precision photography, oscillation photography and diffractometer data collection. Se-Met multiwavelength anamalous dispersion data.

Once the three-dimensional structure of a protein-ligand complex formed between a compound of the present invention and cyclin A is determined, a candidate compound may be examined through the use of computer modelling using a docking program such as GRAM, DOCK or AUTODOCK [Dunbrack et al., 1997, Folding & Design 2:R27-42]. This procedure can include computer fitting of candidate compounds to the ligand binding site to ascertain how well the shape and the chemical structure of the candidate compound will complement the binding site [Bugg et al., Scientific American, December:92-98 (1993); West et al;1 TIPS, 16:67-74 (1995)]. Computer programs can also be employed to estimate the attraction, repulsion and steric hindrance of the two binding partners (i.e. the ligand-binding site and the candidate compound). Generally the tighter the fit, the lower the steric hindrances, and the greater the attractive forces, the more potent the potential drug since these properties are consistent with a tighter binding constant. Furthermore, the more specificity in the design of a potential drug the more likely that the drug will not interact as well with other proteins. This will minimize potential side-effects due to unwanted interactions with other proteins.

Initially candidate compounds can be selected for their structural similarity to a compound of the present invention. The structural analogue can then be systematically modified by computer modelling programs or by inspection until one or more promising candidate compounds are identified. A candidate compound could be obtained by initially screening a random peptide library produced by recombinant bacteriophage for example [Scott and Smith, Science, 249:386-390 (1990); Cwirla et al., Proc. Natl. Acad. Sci., 87:6378-6382 (1990); Devlin et al., Science, 249:404-406 (1990)]. A peptide selected in this manner would then be systematically modified by computer modelling programs as described above, and then treated analogously to a structural analogue as described below.

Once a candidate compound is identified it can be either selected from a library of chemicals as are commercially available or alternatively the candidate compound or antagonist may be synthesized de novo. As mentioned above, the de novo synthesis of one or even a relatively small group of specific compounds is reasonable in the art of drug design. The candidate compound can be placed into a standard binding assay with cyclin A together with a compound of the present invention and its relative activity assessed.

In such an assay, cyclin A may be attached to a solid support. Methods for placing such a binding domain on the solid support are well known in the art and include such things as linking biotin to the ligand binding domain and linking avidin to the solid support. The solid support can be washed to remove unreacted species. A solution of a labelled candidate compound alone or together with a peptide of the present invention can be contacted with the solid support. The solid support is washed again to remove the candidate compound/peptide not bound to the support. The amount of labelled candidate compound remaining with the solid support and thereby bound to the ligand binding domain may be determined. Alternatively, or in addition, the dissociation constant between the labelled candidate compound and cyclin A can be determined. Alternatively, if a compound of the present invention is used, it may be labelled and the decrease in bound labelled compound used an indication of the relative activity of the candidate compound. Suitable labels are exemplified in our WO00/50896 (the contents of which are hereby incorporated by reference) which describes suitable fluorescent labels for use in fluorescent polarisation assays for protein/protein and protein/non-protein binding reactions. Such assay techniques are of use in the assays and methods of the present invention.

When suitable candidate compounds are identified, a supplemental crystal may be grown comprising a protein-candidate complex formed between cyclin A and the potential drug. Preferably the crystal effectively diffracts X-rays for the determination of the atomic coordinates of the protein-candidate complex to a resolution of greater than 5.0 Angstroms, more preferably greater than 3.0 Angstroms, and even more preferably greater than 2.0 Angstroms. The three-dimensional structure of the supplemental crystal may be determined by Molecular Replacement Analysis. Molecular replacement involves using a known three-dimensional structure as a search model to determine the structure of a closely related molecule or protein-candidate complex in a new crystal form. The measured X-ray diffraction properties of the new crystal are compared with the search model structure to compute the position and orientation of the protein in the new crystal. Computer programs that can be used include: X-PLOR (Bruger X-PLOR v.3.1 Manual, New Haven: Yale University (1993B)) and AMORE [J. Navaza, Acta Crystallographics ASO, 157-163 (1994)]. Once the position and orientation are known an electron density map can be calculated using the search model to provide X-ray phases. Thereafter, the electron density is inspected for structural differences and the search model is modified to conform to the new structure.

Candidates whose cyclin A binding capability has thus been verified biochemically can then form the basis for additional rounds of drug design through structure determination, model refinement, synthesis, and biochemical screening all as discussed above, until lead compounds of the desired potency and selectivity are identified. The candidate drug is then contacted with a cell that expresses cyclin A. A candidate drug is identified as a drug when it inhibits CDK2 and/or CDK4 in the cell. The cell can either by isolated from an animal, including a transformed cultured cell; or alternatively, in a living animal. In such assays, and as alternative embodiments of the herein described assays, a functional end-point may be monitored as an indications of efficacy in preference to the detection of cyclin binding. Such end-points include; G0 and/or G1/S cell cycle arrest (using flow cytometry), cell cycle-related apoptosis (sub-G0 population by fluorescence-activated cell sorting, FACS; or TUNEL assay), suppression of E2F transcription factor activity (e.g. using a cellular E2F reporter gene assay), hypophosphorylation of cellular pRb (using Western blot analysis of cell lysates with relevant phospho-specific antibodies), or generally in vitro anti-proliferative effects.

Thus, a further related aspect of the present invention relates to a three dimensional model of a compound of formula I, or variant thereof, as defined above and cyclin A.

The invention further includes a method of using a three-dimensional model of cyclin A and a compound of the present invention in a drug screening assay comprising;
(a) selecting a candidate compound by performing rational drug design with the three-dimensional model, wherein said selecting is performed in conjunction with computer modelling;
(b) contacting said candidate compound with cyclin A, and
(c) detecting the binding of the candidate compound; wherein a potential drug is selected on the basis of the candidate compound having a similar or greater affinity for cyclin A than that of a compound of the invention.

Preferably, the three dimensional model is a computer generated model.

Synthesis

Compounds of general structure I can be prepared by convergent or step-wise assembly of precursors for residues A, B, C, D, and E using any methods known in the art (for recent review refer Ahn, J.-M. et al, 2002, Mini-Rev. Med. Chem., 2, 463). For the formation of a carboxamide (CO—N or N—CO) bond between two residues, the two reaction precursors will contain an amine and carboxyl group, respectively, which groups are condensed using any of the many methods known in peptide chemistry. Similarly, a reduced carboxamide (CH₂—N or N—CH₂) linkage is obtained e.g. by reductive amination of a precursor containing an aldehyde function with a precursor containing an amino function. Sulfonamide (SO₂—N, or N—SO₂) linkages are obtained by condensation of sulfonic acid derivatives, e.g. sulfonyl chlorides, with amines; imine (N═C or C═N) linkages by condensation of aldehydes with amines; semicarbazone (NCONHN═C or C═NNHCON) linkages from condensation between aldehydes and semicarbazides or by action of an alkylisocyanate on a hydrazone (see e.g. Limal, D. et al., 1994, Tetrahedron Lett., 35, 3711); oxime (O—N═C or C═N—O) linkages from condensation of aldehydes or ketones with hydroxylamines (see e.g. Rose, K., 1994, J. Am. Chem. Soc., 116, 30); and ethanolamine (C(OH)CH₂—N or N—CH₂C(OH)) linkages by reaction of epoxides with amines (see e.g. Bennett, F. et al., 1993, Synlett, 703). During the assembly reactions between precursors of peptides or compounds I those functional groups not participating in formation of the desired residue linkage but possessing chemical reactivity are blocked temporarily with suitable protective groups; these groups are chosen in such a way as to be removable selectively and unequivocally following formation of the residue linkage(s) (refer Greene, T. W. and Wuts, P. G. M., 1991, Protective groups in organic synthesis, John Wiley & Sons, Inc.). Assembly strategies based on solid supports, e.g. functionalized synthesis resins, can be used for the preparation of protected precursors of compounds I. In this case any functional group present in any of the precursors is reversibly linked to suitably functionalized solid supports; subsequent coupling reactions are then performed using solid-phase chemistry methods (see e.g. Früchtel, J. S. and Jung, G., 1996, Angew. Chem. Int. Ed. Engl., 35, 17).

The present invention is further described by way of Example and with reference to FIG. 1 which shows the key to the structure abbreviations in Table 1.

### EXAMPLES

### General Synthetic Procedures

Peptides and compounds were assembled using either an ACT 396 automated synthesizer, or an ABI 433A peptide synthesiser. All peptides were assembled on Rink amide resin (Rink, H., 1987, Tetrahedron Lett., 28, 3787). Amino acid, HBTU (2-(1-H-benzotriazol-1-yl)-1,1,3,3-tetramethyluronium hexafluorophosphate), and DIEA (N,N-diisopropylethylamine) solutions were all used at 0.5 M in DMF (N,N-dimethylformamide); piperidine solution was used at 20% in DMF. All washing steps were performed using DMF. Assembly of peptides was performed by standard methods using Fmoc (9-fluorenylmethyloxycarbonyl) methodology (Chan, W. C. and White, P. D. Fmoc Solid Phase Peptide Synthesis; A Practical Approach, Oxford University Press, 2000), using amino acids side-chain protected as Asp(OtBu), Glu(OtBu), Asn(Trt), Gln(Trt), His(Trt), Lys(Boc), Ser(tBu), or as appropriate. After completion of synthesis, resins were dried and peptides were cleaved by treatment with 5:5:90 TIS (triisopropylsilane): H₂O :TFA (trifluoroacetic acid) (Pearson, D. A. et al., 1989, Tetrahedron Lett., 30, 2739), followed by drying in vacuo. Purification was performed using either reversed-phase silica C₈ solid-phase extraction (SPE) cartridges, loading in 0.1% aq TFA, eluting with 60% MeCN/0.1% TFA in H₂O, or by preparative RP-HPLC (MeCN—0.1% aq TFA gradients). Analysis was performed using RP-HPLC, and identity confirmed by mass spectrometry (ES, Micromass), refer Table 2.

### Example 1

### Compounds Containing N-terminal Non-amino Acid Residues (1-23 in Table 1)

All precursors A¹-A¹⁵ in FIG. 1 were obtained commercially as carboxylic acids. Peptides were assembled by standard Fmoc methodology, and incorporation of the N-terminal aryl carboxylic acids was performed using solutions of 100 mg appropriate acid in DMSO (dimethylsulfoxide; 1 mL, 0.5 mL volume used) using HBTU as coupling agent. After synthesis was complete, resins were washed thoroughly with DMF and DCM (dichloromethane), and dried. Cleavage was performed using TIS:H₂O:TFA (1.5 mL volume, 0.5 mL wash), and after dilution with water (1 mL), all solvents were removed in vacuo. Purification was performed using SPE cartridge methods, samples prepared in 2 mL 10% aq DMSO solutions, washed with 0.1% aq TFA and eluted in 60% MeCN/0.1% aq TFA. Analysis was performed using RP-HPLC and mass spectrometry, refer Table 2.

### Example 2

### Compounds Containing C-terminal Non-amino Acid Residues (24-30 in Table 1)

The peptide sequence Boc-Arg(Pbf)-Arg(Pmc)-Leu-OH was assembled on Leu-chlorotrityl resin (Barlos, K. et al., 1991, Int. J Peptide Protein Res., 37, 513) using standard Fmoc methodology. After assembly was complete, the resin was washed, dried and treated with 10% 2,2,2-trifluoroethanol/DCM solution. The protected peptide was recovered by evaporation. Coupling of the arylamine groups E¹-E⁵ in FIG. 1 was performed by treating the protected peptide with 2 mol eq each of 0.5 M HBTU solution and 0.5 M DIPEA, followed by addition of 2 mol eq of the arylamine. Mixtures were stirred overnight, followed by evaporation and treatment with 5:5:90 TIS:H₂O:TFA for I h. Target compounds were recovered by precipitation in diethyl ether (0° C.), followed by drying and purification using RP-HPLC. Identities were confirmed by MS (Table 2).

The peptide sequence Boc-Arg(Pbf)-Arg(Pmc)-Leu-OH was assembled on Weinreb amide resin (N-methoxy-Fmoc-βAla-aminomethylpolystyrene) by standard Fmoc methodology, and the free peptidyl aldehyde was recovered by treatment with 1 M lithium aluminium hydride in tetrahydrofuran solution, followed by quenching with aq citric acid solution, filtration, and extraction into ethyl acetate (Fehrentz, J.-A. et al., 1995, Tetrahedron Lett., 36, 7871). The aldehyde was then treated with 1.5 mol eq of the appropriate arylamine in 1% AcOH/DCM in the presence of polystyrene-immobilized cyanoborohydride (nominally 3 mol eq) (Ley, S. V. et al., 1998, J. Chem. Soc. Perkin Trans. 1, 2239). After mixing overnight, the reactions were filtered and the solvents removed. The residues were treated with 5:5:90 TIS:H₂O:TFA for 1 h. Products were recovered by precipitation in diethyl ether (0° C.), followed by drying and purification using RP-HPLC. Identities were confirmed by MS (Table 2).

### Example 3

### Biological Assays

### Competitive Binding Assay

This assay was performed using half-area black 96-well microtitre plates. To each well were added: 10 μL assay buffer (25 mM HEPES pH 7, 10 mM NaCl, 0.01% Nonidet P-40, 1 mM dithiothreitol), 10 μL test compound solution (in 10% aq DMSO), 10 μL CDK2/cyclin A (ca. 2 μg purified recombinant human kinase complex) in assay buffer, and 10 μL tracer peptide solution (150 nM fluorescein-Ahx-His-Ala-Lys-Arg-Arg-Leu-Ile-Phe-NH₂; refer McInnes, C. et al., 2003, Curr. Med. Chem. Anti-Cancer Agents, 3, 57; Atkinson, G. E. et al., 2002, Bioorg. Med. Chem. Lett., 12, 2501) in assay buffer. After incubation with shaking for 1 h at room temperature, fluorescence polarisation at 485-520 nm was measured using a Tecan Ultra reader. Half-maximal inhibition (IC₅₀) was calculated from dose—response curves.

Functional Kinase Assay

CDK2/cyclin A kinase assays (phosphorylation of natural retinoblastoma protein (pRb)) were performed in 96-well plates using recombinant proteins. To each well were added: 10 μL assay buffer (50 mM HEPES pH 7.4, 20 mM β-glycerophosphate, 5 mM EGTA, 2 mM dithiothreitol, 1 mM NaVO₃, and 20 mM MgCl₂), 5 μL GST-pRb(773-928) substrate stock solution,10 μL test compound solution, 10 μL (2-5 μg protein) of purified recombinant human CDK2/cyclin A stock. The reaction was initiated by addition of 10 μL/well Mg/ATP mix (15 mM MgCl₂, 100 μM ATP with 30-50 kBq per well of [γ-³²P]-ATP) and mixtures were incubated with shaking for 30 min at 30° C. Reactions were stopped on ice, followed by addition of 5 μL/well of glutathione-Sepharose 4B (Amersham Biosciences) and further incubation with shaking for 30 min at room temperature. The mixtures were then filtered on Whatman GF/C filterplates and washed 4 times with 0.2 mL/well of 50 mM HEPES containing 1 mM ATP. Plates were dried, sealed, and scintillant (Microscint 40) was added. Incorporated radioactivity was measured using a scintillation counter (TopCount, Packard Instruments, Pangbourne, Berks, UK). Half-maximal inhibition (IC₅₀) was calculated from dose—response curves.

Results with example compounds from both assays are summarized in Table 3.

Various modifications and variations of the described methods of the invention will be apparent to those skilled in the art without departing from the scope and spirit of the invention. Although the invention has been described in connection with specific preferred embodiments, various modifications of the described modes for carrying out the invention which are obvious to those skilled in the relevant art are intended to fall within the scope of the following claims.

TABLE 1
Example compounds (residues are linked by carboxamide bonds).

| Compound | | Position | | | | |
|---|---|---|---|---|---|---|
| No. | N-terminus | 2 | 3 | 4 | 5 | C-terminus |
| 1 | A¹ | Arg | Leu | Asn | p-F-Phe | NH₂ |
| 2 | A⁴ | Arg | Leu | Asn | p-F-Phe | NH₂ |
| 3 | A⁵ | Arg | Leu | Asn | p-F-Phe | NH₂ |
| 4 | A⁶ | Arg | Leu | Asn | p-F-Phe | NH₂ |
| 5 | A¹¹ | Arg | Leu | Asn | p-F-Phe | NH₂ |
| 6 | A⁷ | Arg | Leu | Asn | p-F-Phe | NH₂ |
| 7 | A⁸ | Arg | Leu | Asn | p-F-Phe | NH₂ |
| 8 | A¹² | Arg | Leu | Asn | p-F-Phe | NH₂ |
| 9 | A² | Arg | Leu | Asn | p-F-Phe | NH₂ |
| 10 | A⁹ | Arg | Leu | Asn | p-F-Phe | NH₂ |
| 11 | A³ | Arg | Leu | Asn | p-F-Phe | NH₂ |
| 12 | A¹³ | Arg | Leu | Asn | p-F-Phe | NH₂ |
| 13 | A¹⁴ | Arg | Leu | Asn | p-F-Phe | NH₂ |
| 14 | A¹⁰ | Arg | Leu | Asn | p-F-Phe | NH₂ |

| | | | | | |
|---|---|---|---|---|---|
| 15 | A¹⁵ | Leu | Asn | p-F-Phe | NH₂ |

| | | | | | |
|---|---|---|---|---|---|
| 16 | A⁹ | Arg | βLeu | p-F-Phe | NH₂ |
| 17 | A⁹ | Lys | βLeu | p-F-Phe | NH₂ |
| 18 | A⁹ | 4-(Gu) Phe | βLeu | p-F-Phe | NH₂ |
| 19 | A⁹ | DMLys | βLeu | p-F-Phe | NH₂ |
| 20 | A⁹ | PipAla | βLeu | p-F-Phe | NH₂ |
| 21 | A⁹ | PipGly | βLeu | p-F-Phe | NH₂ |
| 22 | A⁹ | PipGly | βLeu | p-F-Phe | NH₂ |
| 23 | A⁹ | PipGly | βLeu | p-F-Phe | NH₂ |

| | | | | |
|---|---|---|---|---|
| 24 | Arg | Arg | Leu | E¹ |
| 25 | Arg | Arg | Leu | E² |
| 26 | Arg | Arg | Leu | E³ |
| 27 | Arg | Arg | βLeu | E¹ |
| 28 | Arg | Arg | βLeu | E² |

| | | | |
|---|---|---|---|
| 29 | Arg | Arg | E⁴ |
| 30 | Arg | Arg | E⁵ |

TABLE 2
Mass spectrometric analysis of compounds in Table 1.

| | Structure | | [M + H]⁺ |
|---|---|---|---|
| No. | Formula | MW | observed |
| 1 | C₃₉H₄₇N₁₁O₆FCl | 820.3 | 820.5 |
| 2 | C₃₆H₄₈N₁₂O₇FCl₂ | 850.7 | 849.3 |
| 3 | C₃₅H₄₇N₁₂O₆FCl | 786.3 | 785.3 |
| 4 | C₃₅H₄₅N₁₂O₆FCl₂ | 819.7 | 819.3 |
| 5 | C₃₅H₄₈N₁₁O₆FS | 769.9 | 770.3 |
| 6 | C₃₅H₄₆N₁₂O₆F₂ | 768.8 | 769.1 |
| 7 | C₃₅H₄₇N₁₃O₈F | 796.8 | 796.1 |
| 8 | C₃₄H₄₅N₁₃O₈F | 782.8 | 782.3 |
| 9 | C₄₀H₅₀N₁₁O₇F | 815.9 | 816.4 |
| 10 | C₃₅H₄₅N₁₂O₆FCl₂ | 819.7 | 819.1 |
| 11 | C₄₀H₄₉N₁₁O₇FCl | 850.3 | 850.3 |
| 12 | C₃₇H₄₆N₁₁O₆FS | 791.9 | 792.5 |
| 13 | C₃₇H₄₉N₁₁O₆F₄ | 819.9 | 820.5 |
| 14 | C₃₆H₄₆N₁₂O₆F₄ | 818.8 | 821.7 |
| 15 | C₃₁H₄₂N₇O₇F | 643.7 | 644.3 |
| 16 | C₃₂H₄₁N₁₀O₄FCl₂ | 719.6 | 719.3 |
| 17 | C₃₂H₄₀N₇O₅FCl₂ | 692.6 | 691.2 |
| 18 | C₃₆H₄₁N₁₀O₄FCl₂ | 767.6 | 767.3 |
| 19 | C₃₄H₄₅N₈O₄FCl₂ | 719.6 | 719.40 |
| 20 | C₃₄H₄₃N₈O₄FCl₂ | 717.6 | 717.42 |
| 21 | C₃₃H₄₁N₈O₄FCl₂ | 703.6 | 703.48 |
| 22 | C₃₄H₃₇N₈O₄FCl₂ | 711.6 | 709.40 |
| 23 | C₃₂H₃₆N₉O₄FCl₂ | 700.5 | 700.34 |
| 24 | C₃₀H₄₆N₁₀O₄ | 610.8 | 613.7 |
| 25 | C₃₁H₄₈N₁₀O₄ | 624.8 | 628.5 |
| 26 | C₃₁H₄₈N₁₀O₄ | 624.8 | 625.2 |
| 27 | C₃₁H₄₈N₁₀O₄ | 624.7 | 626.65 |
| 28 | C₃₂H₅₀N₁₀O₄ | 638.8 | 638.36 |
| 29 | C₃₀H₄₈N₁₀O₃ | 596.8 | 597.6 |
| 30 | C₃₁H₅₀N₁₀O₃ | 610.8 | 611.5 |

TABLE 3
Biological activity of compounds in Table 1.

Inhibitory activity
IC₅₀ ± SD (μM)

| | Competitive | Functional kinase |
|---|---|---|
| No. | binding assay | assay |
| 1 | 12 ± 2 | 53 ± 25 |
| 2 | 0.93 ± 0.05 | 11 ± 7 |
| 3 | 4.1 ± 1.3 | 4.3 ± 2.1 |
| 4 | 3.6 ± 0.6 | 6.4 ± 0.3 |
| 5 | 5.8 ± 1.6 | 19 ± 3 |
| 6 | 6.1 ± 1.2 | 4.4 ± 0.8 |
| 7 | 6.4 ± 1.8 | 5.4 ± 0.9 |
| 8 | 28 ± 4 | 26 ± 5 |
| 9 | 15 ± 2 | 37 ± 6 |
| 10 | 2.1 ± 0.2 | 3.7 ± 0.2 |
| 11 | 12 ± 2 | 30 ± 16 |
| 12 | 4.8 ± 0.4 | 15.3 ± 3.1 |
| 13 | 2.6 ± 0.4 | 21.1 ± 7.0 |
| 14 | 2.8 ± 0.42 | 3.65 ± 0.92 |
| 15 | 219 ± 10 | 87 ± 14 |
| 16 | 4.98 ± 0.63 | 5.06 ± 0.81 |
| 17 | 11.46 ± 1.31 | 13.25 ± 0.33 |
| 18 | 5.07 ± 0.84 | 5.30 ± 0.40 |
| 19 | 12.43 ± 0.18 | 13.63 ± 0.63 |
| 20 | 31.54 ± 0.13 | 49.07 ± 16.48 |
| 21 | 22.20 ± 1.04 | 23.55 ± 1.57 |
| 22 | 22.22± | >22.22± |
| 23 | 19.39± | >22.22± |
| 24 | 56.9 ± 1.9 | 21.9 ± 4.9 |
| 25 | 35 ± 2.03 | 29.8 ± 12 |
| 26 | 4.7 ± 0.7 | 8.81 ± 1.08 |
| 27 | 129.5 ± 26.6 | 164.7± |
| 28 | 65 ± 10.7 | 66.4± |
| 29 | 22.7 ± 2.72 | 8.55 ± 2.11 |
| 30 | 58.2 ± 3.82 | 8.37 ± 4.24 |

1. A compound of formula I, or a variant thereof,
A-(B)ₘ-C-(D)ₙ-E (I)
wherein m and n are each independently 0 or 1 and A, B, C, D and E are each independently linked to the respective adjacent residue by a linker group independently selected from carboxamide (CO—N or N—CO), reduced carboxamide (CH₂—N or N—CH₂), sulfonamide (SO₂—N or N—SO₂), imine (N═C or C═N), semicarbazone (NCONHN═C or C═NNHCON), oxime (O—N═C or C═N—O) and ethanolamine (C(OH)CH₂—N or N—CH₂C(OH));
A is
(i) a natural or unnatural amino acid residue having a side chain comprising at least one H-bond acceptor moiety and at least one H-bond donor moiety, or a derivative thereof; or
(ii) R(CO), wherein R is a C₁-C₂₄ hydrocarbyl group comprising at least one H-bond acceptor moiety and optionally one or more H-bond donor moieties, and where R optionally contains one or more heteroatoms selected from S, O, and N, and is optionally substituted by one or more substituents selected from halogen, OMe, CN, CF₃, and NO₂;
each of B and D is independently an amino acid residue selected from arginine, 4-(guanidinyl)phenylalanine (4-(Gu)Phe), piperidinylglycine (PipGly), piperidinylalanine (PipAla), pyridinylalanine, histamine, N,N-(dimethyl) lysine (DMLys), citrulline, glutamine, serine, lysine, asparagine, isoleucine and alanine, or a derivative thereof;
C is NH—X—CO, where X is a C₁-C₄ alkylene group substituted by a straight-chain or branched C₁-C₆ alkylene group, said C₁-C₆ alkylene group optionally containing a H-bond donor or H-bond acceptor moiety;
E is
(j) a natural or unnatural amino acid residue having an aryl or heteroaryl side chain, or a derivative thereof; or
(iii) NHR′, where R′ is a C₁-C₂₄ hydrocarbyl group, optionally containing one or more heteroatoms selected from N, O, and S, and optionally comprising one or more H-bond acceptor or donor moieties; said hydrocarbyl group further comprising a pendent C₄-C₁₂ aryl or heteroaryl group, which itself may be optionally substituted by one or more substituents selected from a H-bond donor moiety, a H-bond acceptor moiety, a halogen, Me, Et, ⁱPr, CF₃, CN and NO₂;
wherein at least one of A and E is other than a natural or unnatural amino acid residue when A, B, C, D and E are each linked to the respective adjacent residue by a carboxamide group.

2. A compound according to claim 1, wherein A, B, C, D and E are each linked to the respective adjacent residue by a carboxamide group.

3. A compound according to claim 1, wherein the H-bond donor moiety is a functional group containing an N—H or O—H group, and the H-bond acceptor moiety is functional group containing C═O or N.

4. A compound according to claim 1, wherein A is R(CO) and R optionally contains up to six heteroatoms, and is optionally substituted by up to six substituents selected from halogen, CN, CF₃, and NO₂.

5. A compound according to claim 1, wherein A is R(CO) and R is cycloalkyl, (CH₂O)ₓ-aryl or (CH₂O)ₓ-heteroaryl, and x is 0 or 1, wherein said cycloalkyl, aryl or heteroaryl group may be optionally substituted by one or more substituents selected from
NO₂;
halogen;
alkyl;
CF₃;
2-imidazolidinethione;
NH(CO)-heteroaryl, aryl or heteroaryl, each of which may be optionally substituted by one or more substituents selected from halogen, alkyl, NO₂, CF₃ and alkoxy.

6. A compound according to claim 5, wherein the heteroaryl group is selected from 1,2,4-triazole, benzothiazole, benzimidazole, pyrrole, isooxazole and imidazo[1,2-a]pyridine.

7. A compound according to claim 1, wherein A is selected from the following:

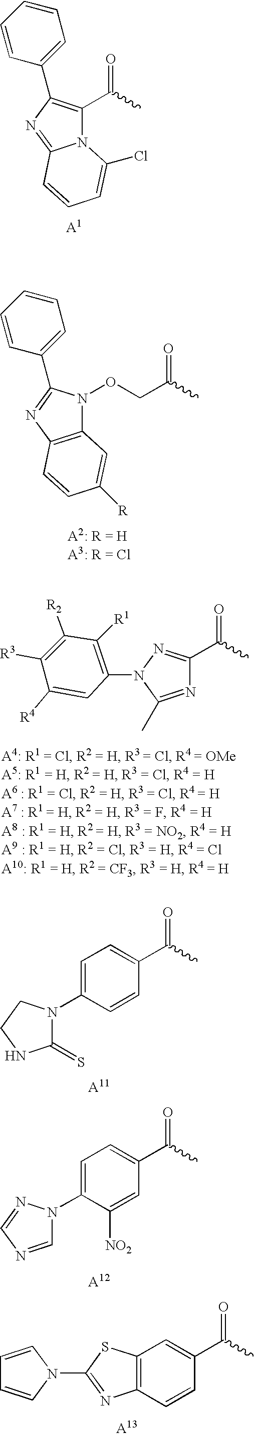

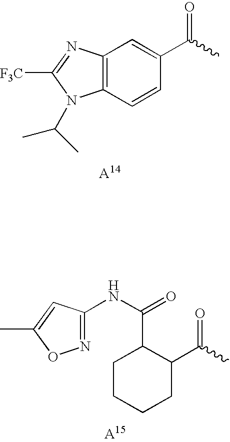

8. A compound according to claim 1, wherein E is NHR′ and the hydrocarbyl group of E optionally contains up to six heteroatoms, and optionally comprises up to two H-bond acceptor or donor moieties, and wherein the pendant C₁-C₁₂ aryl or heteroaryl group is optionally substituted by up to four substituents selected from a H-bond donor moiety, a H-bond acceptor moiety, a halogen, Me, Et, ⁱPr, CF₃, CN and NO₂.

9. A compound according to claim 8, wherein E is NHR′ and R′ is [CH(R^{a})CH₂NH]ₚ[CH₂]_{q}Ar^{a}[CH₂]ᵣAr^{b}, where R^{a} is a straight or branched chain C₁-C6 alkyl group, p, q and r are each independently 0 or 1, and Ar^{a} and Ar^{b} are each independently aryl groups optionally substituted by one or more substituents selected from halogen, Me, Et, ⁱPr, CF₃, CN and NO₂.

10. A compound according to claim 9, wherein E is 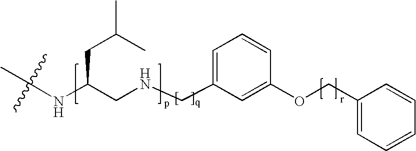
and p, q and are as defined in claim 9.

11. A compound according to claim 10, wherein E is selected from the following:

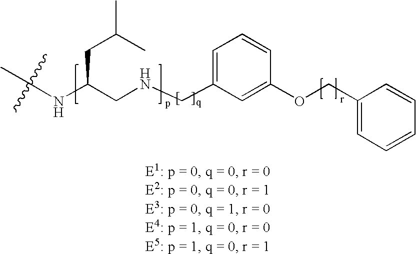

12. A compound according to claim 1, wherein C is selected from alanine, valine, leucine, β-leucine, β-OH-β-leucine, isoleucine, aspartate, glutamate, asparagine, glutamine, lysine, arginine, serine and threonine.

13. A compound according to claim 12, wherein C is selected from leucine, isoleucine, β-leucine, β-OH-β-leucine, and asparagine;

14. A compound according to claim 13, wherein C is leucine or β-leucine.

15. A compound according to claim 1, wherein B is selected from arginine, 4-(guanidinyl)phenylalanine (4-(Gu)Phe), piperidinylglycine (PipGly), piperidinylalanine (PipAla), pyridinylalanine, histamine, N,N-(dimethyl) lysine (DMLys), citrulline, glutamine, serine and lysine.

16. A compound according to claim 15, wherein B is arginine.

17. A compound according to claim 1, wherein D is selected from asparagine, isoleucine and alanine.

18. A compound according to claim 17, wherein D is asparagine.

19. A compound according to claim 1, wherein A is selected from arginine, glutamine, citrulline.

20. A compound according to claim 1, wherein E is selected from phenylalanine, para-fluorophenylalanine, meta-fluorophenylalanine, ortho-chlorophenylalanine, para-chlorophenylalanine, meta-chorophenylalanine, thienylalanine, N-methylphenylalanine, homophenylalanine (Hof), tyrosine, tryptophan, 1-naphthylalanine (1Nal), 2-naphthylalanine (2Nal) and biphenylalanine (Bip) or (Tic).

21. A compound according to claim 20, wherein E is selected from phenylalanine, para-fluorophenylalanine, meta-fluorophenylalanine, ortho-chlorophenylalanine, para-chlorophenylalanine, meta-chorophenylalanine, thienylalanine and N-methylphenylalanine.

22. A compound according to claim 21, wherein E is para-fluorophenylalanine

23. A variant of a compound according to claim 1, wherein:
(a) A is unchanged or conservatively substituted;
(b) B is substituted by any amino acid capable of providing at least one site for participating in hydrogen bonding;
(c) C is unchanged or conservatively substituted;
(d) D is unchanged or conservatively substituted;
(e) E is unchanged or substituted by any aromatic amino acid.

24. A compound according to claim 1, wherein m and n are both 1.

25. A compound according to claim 1, wherein m is 1 and n is 0.

26. A compound according to claim 1, wherein m is 0 and n is 1.

27. A compound according to claim 1, wherein m and n are both 0.

28. A compound according to claim 1, which is selected from the following:

| Compound | | | | | | |
|---|---|---|---|---|---|---|
| No. | N-terminus | | | | | C-terminus |
| 1 | A¹ | Arg | Leu | Asn | p-F-Phe | NH₂ |
| 2 | A⁴ | Arg | Leu | Asn | p-F-Phe | NH₂ |
| 3 | A⁵ | Arg | Leu | Asn | p-F-Phe | NH₂ |
| 4 | A⁶ | Arg | Leu | Asn | p-F-Phe | NH₂ |
| 5 | A¹¹ | Arg | Leu | Asn | p-F-Phe | NH₂ |
| 6 | A⁷ | Arg | Leu | Asn | p-F-Phe | NH₂ |
| 7 | A⁸ | Arg | Leu | Asn | p-F-Phe | NH₂ |
| 8 | A¹² | Arg | Leu | Asn | p-F-Phe | NH₂ |
| 9 | A² | Arg | Leu | Asn | p-F-Phe | NH₂ |
| 10 | A⁹ | Arg | Leu | Asn | p-F-Phe | NH₂ |
| 11 | A³ | Arg | Leu | Asn | p-F-Phe | NH₂ |
| 12 | A¹³ | Arg | Leu | Asn | p-F-Phe | NH₂ |
| 13 | A¹⁴ | Arg | Leu | Asn | p-F-Phe | NH₂ |
| 14 | A¹⁰ | Arg | Leu | Asn | p-F-Phe | NH₂ |
| | | | | | |
|---|---|---|---|---|---|
| 15 | A¹⁵ | Leu | Asn | p-F-Phe | NH₂ |
| | | | | | |
|---|---|---|---|---|---|
| 16 | A⁹ | Arg | βLeu | p-F-Phe | NH₂ |
| 17 | A⁹ | Lys | βLeu | p-F-Phe | NH₂ |
| 18 | A⁹ | 4-(Gu) Phe | βLeu | p-F-Phe | NH₂ |
| 19 | A⁹ | DMLys | βLeu | p-F-Phe | NH₂ |
| 20 | A⁹ | PipAla | βLeu | p-F-Phe | NH₂ |
| 21 | A⁹ | PipGly | βLeu | p-F-Phe | NH₂ |
| 22 | A⁹ | PipGly | βLeu | p-F-Phe | NH₂ |
| 23 | A⁹ | PipGly | βLeu | p-F-Phe | NH₂ |
| | | | | | |
|---|---|---|---|---|---|
| 24 | H | Arg | Arg | Leu | E¹ |
| 25 | H | Arg | Arg | Leu | E² |
| 26 | H | Arg | Arg | Leu | E³ |
| 27 | H | Arg | Arg | βLeu | E¹ |
| 28 | H | Arg | Arg | βLeu | E² |
| | | | | |
|---|---|---|---|---|
| 29 | H | Arg | Arg | E⁴ |
| 30 | H | Arg | Arg | E⁵ |

wherein A¹⁻¹⁵ and E¹⁻⁵ are as defined above in claims 7 and 11 respectively, and wherein each residue is linked to the adjacent residue by a carboxamide linker group.

29. A variant of a compound according to claim 1, which is (a) modified by substitution of one or more natural or unnatural amino acid residues by the corresponding D-stereomer; (b) a chemical derivative of the compound; (c) a cyclic compound derived from the compound or derivative thereof; (d) a multimer of said compounds; (e) the D-stereomer form of said compound; or (f) a compound wherein the order of the final two residues at the C-terminal end are reversed.

30. A pharmaceutical composition comprising a compound according to claim 1 admixed with a pharmaceutically acceptable diluent excipient or carrier.

31. A method of treating a proliferative disorder, comprising administering to a subject in need thereof, a compound according to claim 1, such that the subject is treated for the proliferative disorder.

32. An assay for identifying candidate substances capable of binding to a cyclin associated with a G1 control CDK enzyme and/or inhibiting said enzyme, comprising;
(a) bringing into contact a compound of claim 1, said cyclin, said CDK and said candidate substance, under conditions wherein, in the absence of the candidate substance being an inhibitor of interaction of the cyclin/CDK interaction, the compound would bind to said cyclin, and
(b) monitoring any change in the expected binding of the compound and the cyclin.

33. An assay for the identification of compounds that interact with a cyclin or a cyclin when complexed with the physiologically relevant CDK, comprising:
(a) incubating a candidate compound and a compound according to claim 1, or a variant thereof, and a cyclin or cyclin/CDK complex,
(b) detecting binding of either the candidate compound or the compound with the cyclin.

34. An assay according to claim 32 or claim 33 wherein the cyclin is selected from cyclin A, cyclin E or cyclin D.

35. An assay according to claim 34, wherein the cyclin is cyclin A.

36. An assay according to any of claims 32 or claim 33, comprising use of a three dimensional model of a cyclin and a candidate compound.

37. An assay according to claim 32 or 33, wherein at least one of the assay components is bound to a solid phase.

38. An assay according to claim 37, wherein the compound is labelled so as to emit a signal when bound to said cyclin.

39. An assay according to claim 37, wherein the cyclin is labelled so as to emit a signal when bound to the compound.

40. An assay according to claim 38, wherein one of the assay components is labelled with a fluorescence emitter and the signal is detected using fluorescence polarisation techniques.

41. A method of using a cyclin in a drug screening assay comprising:
(a) selecting a candidate compound by performing rational drug design with a three-dimensional model of said cyclin, wherein said selecting is performed in conjunction with computer modeling;
(b) contacting the candidate compound with the cyclin; and
(c) detecting the binding of the candidate compound for the cyclin groove; wherein a potential drug is selected on the basis of its having a greater affinity for the cyclin groove than that of a compound according to claim 1.

42. A method or assay according to any of claims 32 or 33, wherein the method of detection comprises monitoring G0 and/or G1/S cell cycle, cell cycle-related apoptosis, suppression of E2F transcription factor, hypophosphorylation of cellular pRb, or in vitro anti-proliferative effects.
